(19) United States
(12) Patent Application Publication
(10) Pub. No.: US 2021/0134408 A1
(43) Pub. Date: May 6, 2021

(54) SYSTEM AND METHOD FOR PATIENT AGGREGATE MEDICAL RECORD ACCESS, CARE PROVIDER MANAGEMENT, AND PATIENT CARE MONITORING/ASSESSMENT

(71) Applicant: METACINE, LLC, Austin, TX (US)

(72) Inventors: Luke Thomas REDMAN, Round Rock, TX (US); Dieter MARTIN, Austin, TX (US)

(21) Appl. No.: 17/044,295

(22) PCT Filed: Mar. 29, 2019
(86) PCT No.: PCT/US19/24932

§ 371 Date: Sep. 30, 2020

Related U.S. Application Data
(60) Provisional application No. 62/650,947, filed on Mar. 30, 2018.

(51) Int. Cl. G16H 10/60; G16H 50/20; G16H 50/30; G16H 40/20

(52) U.S. Cl. CPC G16H 10/60; G16H 40/20; G16H 50/30; G16H 50/20

(57) ABSTRACT

Systems and methods for collecting patient data for a selected group of patients and providing the patient data in an informative manner to medical care providers are provided. The systems and methods can receive patient data from electronic medical records and/or other sources, aggregate the data, and analyze the data to provide the data to medical providers in a usable format. The systems and methods can further analyze the aggregate patient data based on physiological criterion to identify the incidence of triggering events. The systems and methods can further generate a prioritized list of patients for attention by medical providers based on the occurrence of triggering events or other data indicators.

### CROSS-REFERENCE TO RELATED APPLICATIONS

This Application claims priority to U.S. Provisional Patent Application Ser. No. 62/650,947, filed on Mar. 30, 2018, to Luke Thomas Redman et al., entitled “System and Method for Patient Aggregate Medical Record Access, Care Provider Management, and Patient Care Monitoring/Assessment,” currently pending, the entire disclosure of which is incorporated herein by reference.

### FIELD OF THE INVENTION

The disclosure as detailed herein relates to the technical field of systems and methods useful in improving the efficiency and quality of health care administration to a patient, and further useful in monitoring and assessing the efficiency and quality of health care administration to a patient.

### DESCRIPTION OF RELATED ART

Frequently in inpatient care settings, particularly sub-acute settings, a physician does not see every patient daily, and as a consequence patients frequently have declining health status between physician visits that goes unnoticed without appropriate patient intervention.

U.S. Patent Application Publication No. 2017/0046499 to Hu et al. relates generally to methods for predicting patient deterioration or clinical events. The methods provide for predictions of patient deterioration or clinical events by detecting patterns in heterogeneous temporal clinical data streams that are predictive of certain clinical end points and matching the patient state with those patterns. The detected patterns, referred to as SuperAlarm triggers, are described as a predictive combination of frequently co-occurring monitor alarms, conditions and laboratory test results that can predict patient deterioration for imminent life-threatening events. An aim to be achieved is to reduce the number of false alarms and alarm fatigue compared to conventional alarms.

U.S. Patent Application Publication No. 2014/0257853 to Huang relates generally to a health data collection and analysis device that can be used as a means for examining the user and obtaining at least one examination record. An objective described is to provide a device for a user to record medication, examination and activity records for use by health related institutions such as pharmacies when preparing medicine or other health products for clients.

U.S. Patent Application Publication No. 2017/0281095 to An et al. relates generally to systems and methods for monitoring patients with multiple chronic diseases. The system is described as including a health status monitor that receives diagnostic data including physiological signals from a patient, and then producing a first risk indication of the patient developing a first disease and a second risk indication of the patient developing a second different disease. Also generally described is a composite health status indicator and alert for worsening health status of a patient.

U.S. Patent Application Publication No. 2017/0161439 to Raduchel et al. relates generally to systems and methods for providing a healthcare provider with electronic medical records of a patient, and a recommendation, or an alert relating to the patient, based on an analysis of the patient's health data. A mobile device associated with a user is described to aggregate electronic medical records for the user and enable the user to provide the user's electronic medical records to a physician and/or other medical personnel.

Despite these and other teachings, the medical arts remain in need of systems and methods for health care providers and care facilities to improve efficiency of health care delivery to a defined patient/resident population, as well to improve the timeliness and quality of patient care delivery.

### SUMMARY OF THE INVENTION

The present invention is directed generally to systems and methods for collecting and aggregating patient health information for a selected group of patients/residents, and for monitoring patient health and prioritizing patient care based on the identification of triggering events for a patient/resident within a collected group of patient/resident health information.

The system according to one embodiment can include one or more database modules for storing patient data and metrics (e.g., temperature, blood pressure, insulin levels, weight, etc.) for one or more patients, a processing component for carrying out instructions for providing access to patient data and processing/analyzing patient data, and a software program or application configured to make accessible the patient data in various types of configurations and formats through a user interface. In particular, the system of the present invention can be utilized to provide aggregated and processed patient data to physicians and other health care providers to allow such providers to administer improved care to patients and to identify patients in need of urgent care.

According to one embodiment, the system and method are configured to utilize patient data relating to one or more specific physiologically relevant criterion and to analyze the patient data to determine the incidence of triggering events in individual patients within a defined group. The system and method can automatically provide a prioritized list of patients for medical attention based on the analyzed patient data and occurrence of triggering events in individual patients. This can enable physicians and providers to ensure that medical attention is being prioritized based on need, which can result in improved patient care and outcomes, decreased physician visits, and decreased overall medical care costs.

In addition, the present invention is directed to a system and/or method for accessing a patient's record identified to have a selected characteristic symptom and/or characteristic of choice, as among a group of patients in a defined group. In a particular embodiment, the system and method is provided in an electronically accessible format and/or database interface. By way of example, the database interface would be amendable to ready access by a physician or other health care provider, whereby an identified group of informational metrics, such as patient metrics (temperature, blood pressure, insulin levels, weight, etc.) and/or patient medical records, of individuals within the defined group may be screened according to a criteria or set of criteria, of choice.

The described systems and/or methods may be configured so as to be provided as an “app” on an electronic device, such as a personal electronic device (electronic tablet, cellular phone (iphone), desk top computer, laptop computer, etc.).

The systems and methods may also incorporate a computer program. In one embodiment, the computer program comprises a program code means for causing a system and/or method for accessing the defined population of patient data as defined above, and to carry out steps of the method for selecting for the identified metric or group of identified metrics of interest, when the computer program is run on a computer controlling the system and/or method for carrying out the selection and/or screening protocol.

### DESCRIPTION OF FIGURES

FIG. 1 is a schematic representation of a system for providing processed patient data and information to medical providers and personnel in accordance with one embodiment of the present invention;

FIG. 2 is a flowchart of a method for providing processed patient data and information to medical providers and personnel in accordance with one embodiment of the present invention;

FIG. 3 is a flowchart of a method for providing processed patient data and prioritized patient attention information to medical providers and personnel in accordance with one embodiment of the present invention;

FIGS. 4A-4I demonstrate the application of a system and method for monitoring patient care at a skilled nursing facility (“Facility #1”) with a population of approximately 150 individuals in accordance with one embodiment of the present invention;

FIGS. 5A-5I demonstrate the application a system and method for monitoring patient care at another skilled nursing facility (“Facility #2”) with a population of approximately 50 individuals in accordance with one embodiment of the present invention; and

FIGS. 6A-6I demonstrate the application of a system and method for monitoring patient care at yet another skilled nursing facility (“Facility #3”) with a population of approximately 50 individuals in accordance with one embodiment of the present invention wherein the data provided by the system and method was not utilized.

### DETAILED DESCRIPTION OF THE PREFERRED EMBODIMENTS

The invention will now be described with reference to the drawing figures, in which like reference numerals refer to like parts throughout. For purposes of clarity in illustrating the characteristics of the present invention, proportional relationships of the elements have not necessarily been maintained in the drawing figures.

The present invention is directed generally to systems and methods for prioritizing patient care based on one or more triggering criteria relating to patient metrics. The systems and methods of the present invention can be utilized to inform medical care providers, physicians, etc. of patients experiencing stagnation or decline in medical conditions and/or treatment, and can provide in an informative and simplified manner a prioritized list of patients in need of medical attention. For example, according to one embodiment, the systems and methods of the present invention can be utilized to create a prioritized list of patients for a doctor or other medical professional during a visit to a particular medical care facility. In such an embodiment, the prioritized list of patients can inform the medical care provider of the patients that would be most benefited from medical care and allow the medical care provider to ensure patients in greater need of care receive such care in a prioritized manner. The systems and methods of the present invention can further be utilized to generate patient data reports for medical care providers to identify key physiological and health metrics of individual patients and selected groups of patients to assist and inform medical care providers of the current state and changes in patient's conditions.

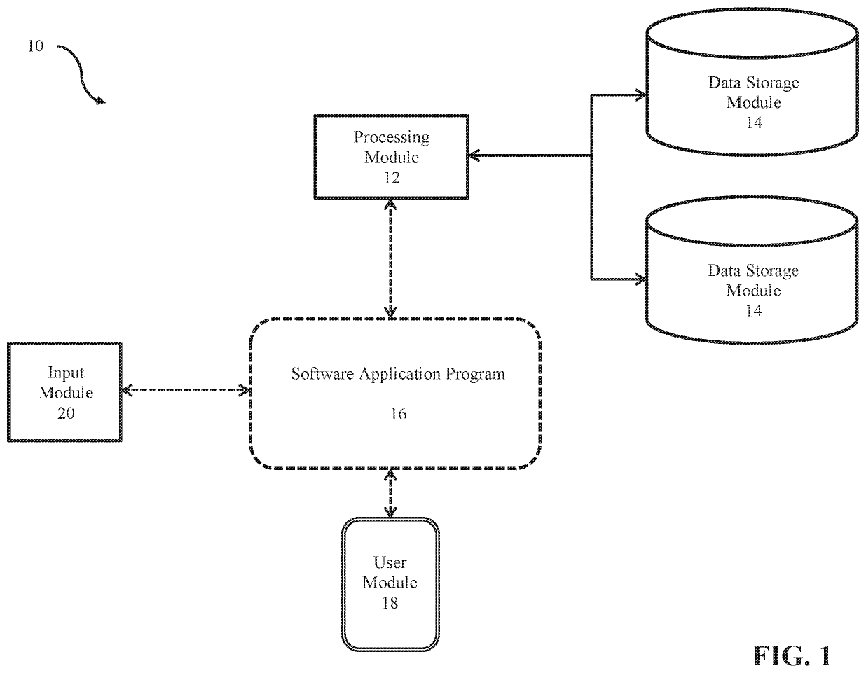

FIG. 1 shows a schematic representation of a system 10, according to one embodiment of the present invention, that is configured to aggregate, process and analyze patient data to enable medical personnel to make more informed decisions and/or prioritize patient care for a selected group of patients. As described herein, system 10 can be utilized in connection with a method 100 to analyze patient data for a selected group of patients and generate reports and information for medical facilities and medical personnel (e.g., physicians and nurse practitioners) to (i) identify patients with increased or urgent needs of medical care, (ii) provide a more informed perspective for individual patients and/or a patient group collectively, (iii) identify and analyze the effectiveness of specific patient therapies, and/or (iv) identify and analyze the quality of care of a medical facility and/or individual medical personnel.

As shown in FIG. 1, system 10 can include a processing module 12, one or more patient data modules or repositories 14, and a software application or program 16. The processing module 12 can be configured as any suitable type of computer processor capable of carrying out one or more sets of instructions. Processing module 12 can be in communication with data modules 14 in order to send, receive and query the modules 14 in order to obtain and reference patient data, metrics and information. According to one embodiment of the present invention, processing module 12 can include application program 16 with programming instructions, that when executed by processing unit 12 cause the system 10 to carry out one or more steps and/or procedures for sending, receiving, processing, and/or storing various types of data and information as described in greater detail below. Processing module 12 via software program 16 can further be configured to display patient data, metrics and information to medical providers and medical personnel. As also shown in FIG. 1, system 10 can be configured for use with a user computer, tablet or other suitable display device 18 that can be utilized in connection with program 16 and display information for medical providers and personnel. Device 18 can be configured as any suitable device, such as a computer, laptop, mobile device, tablet, etc., that can be associated with or otherwise communicate with processing module 12, to carry out one or more sets of instructions and operate software program 16.

Data modules or repositories 14 can be configured as any suitable type of storage component for storing (or otherwise making accessible) various types of data, metrics, and information of patients and patient groups. According to one embodiment, storage modules 14 can include clinical data from electronic medical records (EMRs), data entered by medical personnel on site or through program 16 (such as through an input module 20 as shown in FIG. 1), data automatically recorded through various types of equipment on site (such as through input module 20), and/or other types of patient data. According to certain embodiments, one or more modules 14 can be configured as commercial databases, servers, etc. that make accessible various types of information. According to certain embodiments, one or more modules 14 can be configured to store processed and/or analyzed data created through application 16 and system 10 during use by medical providers, facilities and personnel.

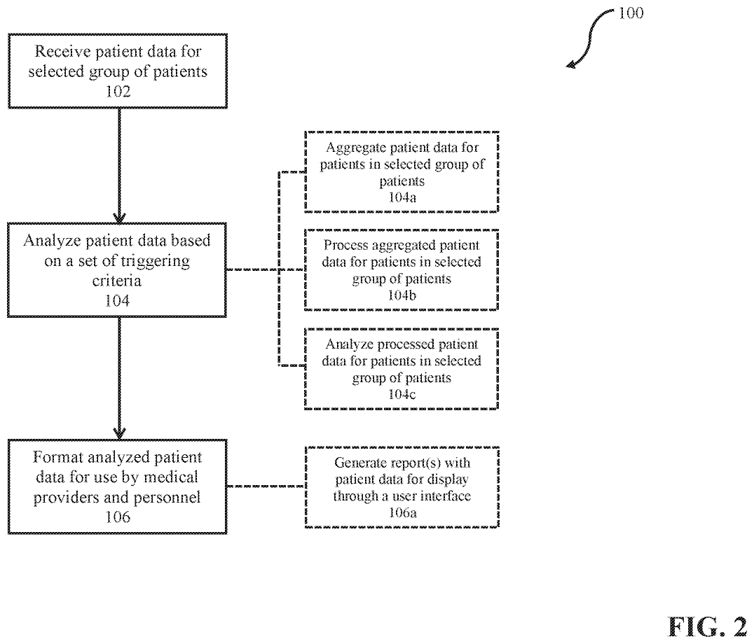

Turning to FIG. 2, system 10 can be utilized in connection with method 100 (according to one embodiment) for collecting, aggregating, analyzing and processing patient data for selected patients and then providing the processed patient data to medical facilities and personnel. Method 100 can be utilized to generate a prioritized patient visit list for medical personnel (e.g., physicians), assess the effectiveness of certain treatments, assess the effectiveness of medical facilities and/or personnel, and/or provide for automated monitoring of patient health metrics, among others uses, as described in greater detail in the examples below.

As shown in FIG. 2, method 100 according to one embodiment can begin with receiving patient data for a selected group of patients (i.e., set of one or more patients) at step 102. The patient data can include raw clinical data, patient metrics, vitals and/or other medically relevant records and data (which can represent patient data at one or more time intervals). According to one embodiment, the patient data can include data representing certain physiologically relevant criterion or metrics (e.g., hypertension, hypotension, tachycardia, bradycardia, hypoglycemia, hyperglycemia, O2 levels, pain (on a scale of 1-10), temperature, etc.).

At step 104, the patient data can be aggregated, processed and/or analyzed based on a defined set of triggering criteria. According to one embodiment, the triggering criteria can be related to certain physiological conditions (hypertension, hypotension, tachycardia, bradycardia, hypoglycemia, hyperglycemia, O2 levels, etc.). At step 104, the patient data received from step 102 can be aggregated together for each patient in the selected group of patients to generate a comprehensive set of patient data for each patient. As described above, this patient data can span one or more time periods to allow for changes in certain patient data to be identified and assessed. This aggregated patient can also be stored for future use in certain embodiments. Also at step 104, the aggregated patient data for each patient can be analyzed to identify the “triggering” occurrence or incidence of specific physiological conditions and/or other health metrics. The analysis and identification of triggering incidences can be accomplished by analyzing patient data relevant to the specific physiological conditions/health metrics and determining whether analyzed patient data exceeds certain criteria or value thresholds (or value change thresholds).

At step 106, the processed/analyzed patient data can be formatted and configured for use by medical care providers and/or medical care personnel (e.g., physicians, etc.). According to one embodiment, at step 104, patient data reports can be generated to provide the processed/analyzed patient data for each patient in a group of patients for viewing by medical care providers and personnel. The reports can identify changes in patient health metrics and physiological conditions and provide the patient data in a condensed and informative format to assist the medical care providers and personnel in assessing the current condition of each patient.

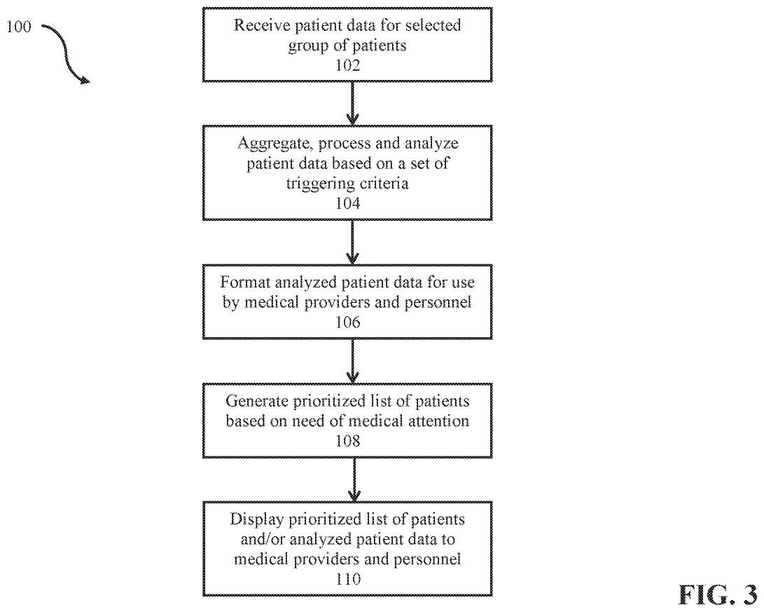

As shown in FIG. 3, according to one embodiment, method 100 can include a step 108 where a stratified and/or prioritized patient chart is generated identifying and organizing patients in a selected group of patients based on the need of current medical attention or treatment. At step 108, the stratified and/or prioritized patient list can be based on the incidence or occurrence of specific physiological conditions identified through the use of the set of triggering criteria utilized at step 104. The stratified/prioritized patient chart and/or analyze patient data can be provided to the desired medical personnel and displayed through a user device (such as computer, table or mobile device 16) as indicated at step 110.

The stratified and/or prioritized patient charts can be provided to medical care providers and/or personnel to identify which patients should be attended to first or the order in which patients should receive medical attention. For example, it is common for a physician to visit a medical care facility to provide attention and/or treatment to one or more patients in a selected group of patients, where the physician does not have the ability to attend to every patient during the visit, and/or where the physician does not have a meaningful way to identify which patients are in most need of medical attention. Method 100 can be utilized to access patient data for the selected group of patients at the medical facility, analyze and process the patient data for each patient, and generate patient reports and a prioritized list of patients to inform the physician of the current medical conditions of the patients and which patients should receive care first and/or most urgently require intervention.

As described above, method 100 can be used in conjunction with system 10 to provide a means for healthcare facilities, providers and personnel to provide effective patient care, assess the health conditions of individual patients and selected groups of patients (such as patients at a particular facility, skilled nursing facility, etc.), and/or assess the effectiveness of particular types of medical treatment, medical facilities, providers or personnel. In such and embodiment, system 10 can be configured as an onsite or remote computer network where application or program 16 (and processing module 12) can be operated through a central computer and/or one or more user devices 18 used by a particular medical facility or personnel. The program 16 can be configured to access database modules 14 to receive patient data and metrics for one or more individuals in a selected group of individuals. The accessed/received data and metrics can include current measurements of one or more selected physiological criterion of the selected group of individuals. The system 10 can subsequently process and/or analyze the patient data based on selected triggering criterion and benchmarks to the current conditions of the individuals. For example, system 10 can identify individuals showing signs of stagnation or decline. The program 16, through user device 18 can then display for a provider (e.g., physician or nurse practitioner) the processed patient data for a specific individual or for the entire group of selected individuals, and/or a list of stratified individuals based on the triggering incidences/benchmarks so the provider has an informed perspective on which individuals most urgently require intervention. This system 10 and program 16 can also have numerous applications in attributing quality of care for a patient to the level of a particular physician, facility, nurse, and so on, for the purposes of identifying which settings/providers consistently provide the best clinical outcomes.

Additional examples of the uses of system 10 and method 100 describing the invention in additional detail are provided below.

### EXAMPLE 1

### Physicians Groups

The present example describes the utility of the systems and methods for use by physicians groups. By way of example, the system 10 and method 100 may be used to:
1. Aggregate the data from multiple electronic medical records and allow for query-based ad hoc analyses, whether manual or automated.
2. Aggregate the data from multiple electronic medical records to create a singular, longitudinal medical record.
3. Remotely analyze patient vital signs taken by inpatient care staff, using care protocols or parameters to programmatically identify outlier patients.
4. Analyze the trending of patient vital signs and the impacts of therapies such as medications.
5. Analyze the clinical outcomes of particular staff, e.g., which physicians' patients are more or less likely to be readmitted to a hospital or have another adverse outcome.
6. Assess and enforce proper provider adherence to clinical best practices and defined quality performance metrics.
7. Assess the current status of performance on clinical quality metrics as part of a performance-based contract, i.e., an ACO.
8. Access patient vital signs to indicate when a billable patient visit is warranted.
9. Analyze the clinical outcomes of inpatient acute care facilities, such as inpatient hospitals or rehabilitation hospitals.
10. Analyze the clinical outcomes of sub-acute care settings, such as skilled nursing facilities or home health agencies.
11. Identify and analyze the particular therapies administered by these agencies, such as physical therapy, respiratory therapy, or speech therapy, or to assess whether these services were rendered as planned.
12. Aggregate and merge data from non-clinical sources, with data from clinical sources, e.g., the integration of physical activity data from a wearable device.

### EXAMPLE 2

### Facility Operators

The present example presents a description of the use of the present tools, systems and methods by a variety of facility operators, such as health care facility operators.

By way of example, facility operators, such as hospitals or skilled nursing facilities (SNF), may employ the presently described system 10 and method 100 in the following applications:
1. To identify differences in clinical outcomes for care rendered by organic staff, e.g. comparing the clinical outcomes of one nurse versus another, or one shift of nurses vs another.
2. To identify differences in clinical outcomes for care rendered by downstream providers, e.g., for a hospital to evaluate which home health agencies provide the best care for patients either generally or with a particular condition.
3. To identify differences in measurement across organic staff, e.g., whether a nurse consistently takes blood pressure measurements incorrectly as validated by that nurse consistently having outlier measurements compared to other nurses.
4. To aid in ‘workforce automation’ by reducing communication burden, i.e., the nurse only needs to enter vitals into the computer and does not need to notify the doctor of an abnormality because that doctor will see the abnormal vitals on his or her dashboard.

### EXAMPLE 3

### Payers or Employers

The present example presents a description of the use of the present tools, systems and methods by a variety of health care cost payers (e.g., insurance companies) or employers.

By way of example, the system 10 and method 100 may be used to:
1. Gain real-time insight into the clinical and vital sign status of beneficiaries currently under the care of an acute or subacute provider, such as a hospital or home health agency, for the purposes of stratifying the performance of facilities.
2. Gain real-time insight into the care delivered by a hospital for the purposes of utilization management.
3. Gain real-time insight into the clinical status of a patient for the purposes of ascertaining if a particular therapy is working insofar as it is resulting in improved vital signs.

### EXAMPLE 4

### Skilled Nursing Home Study #1

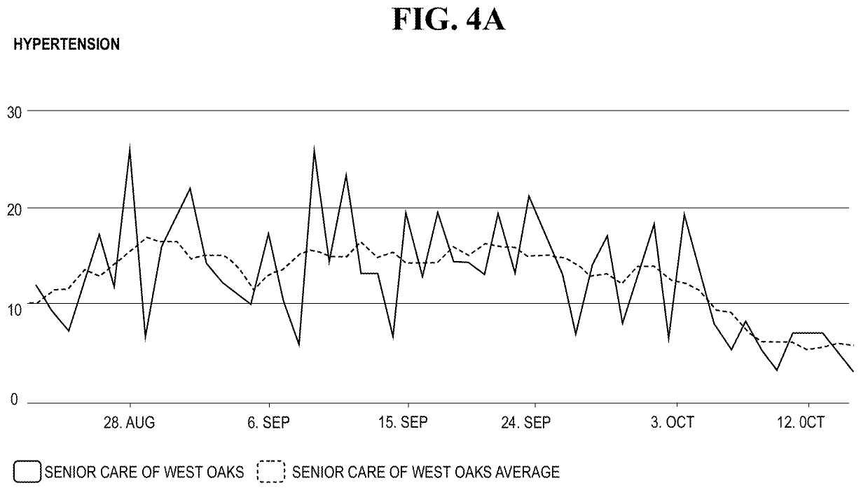

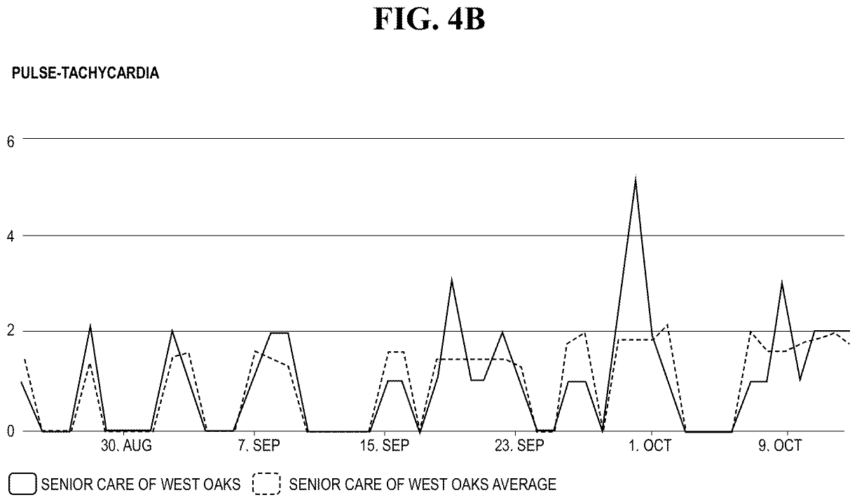

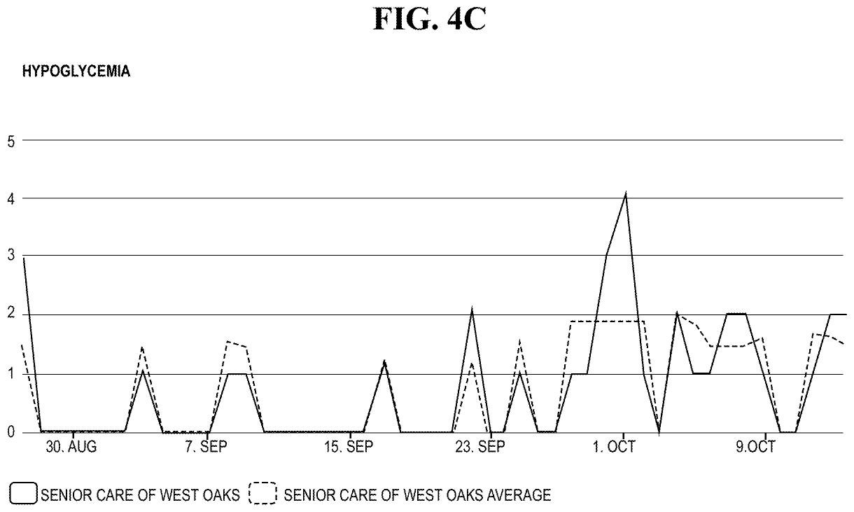

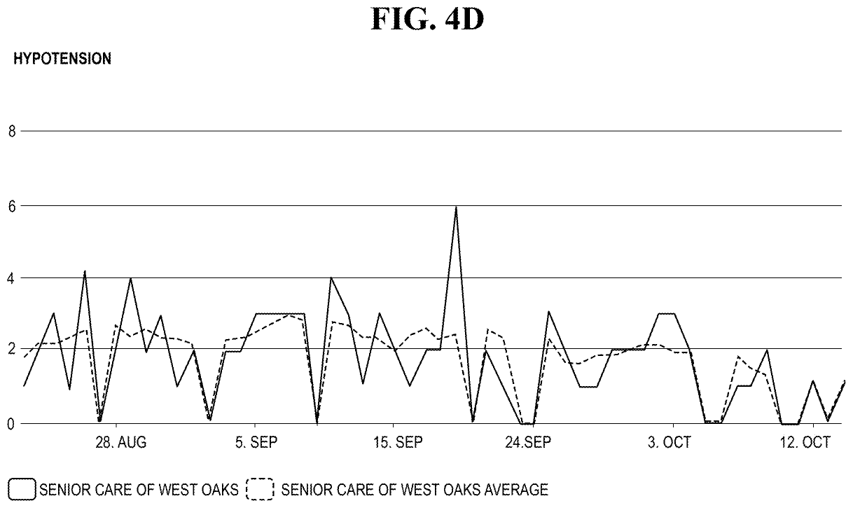

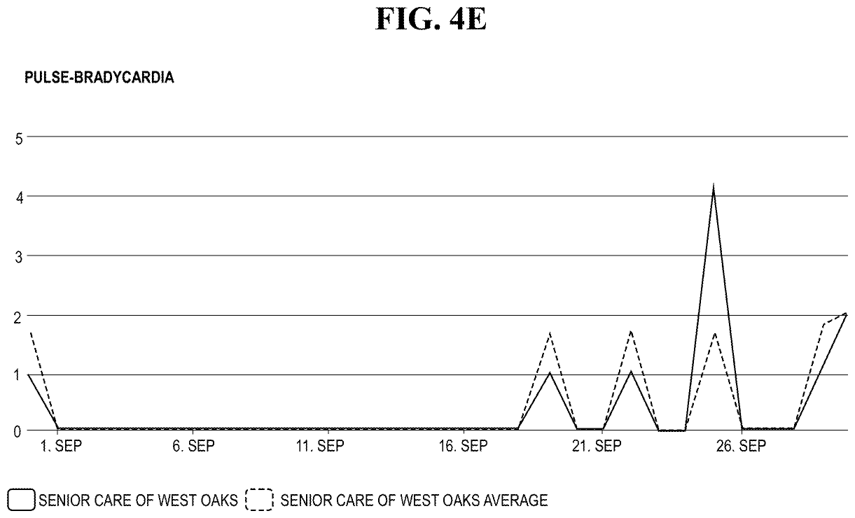

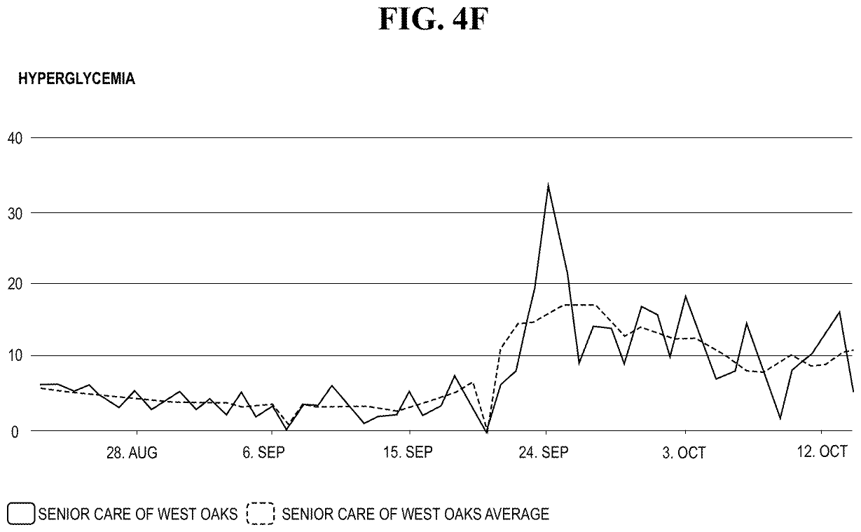

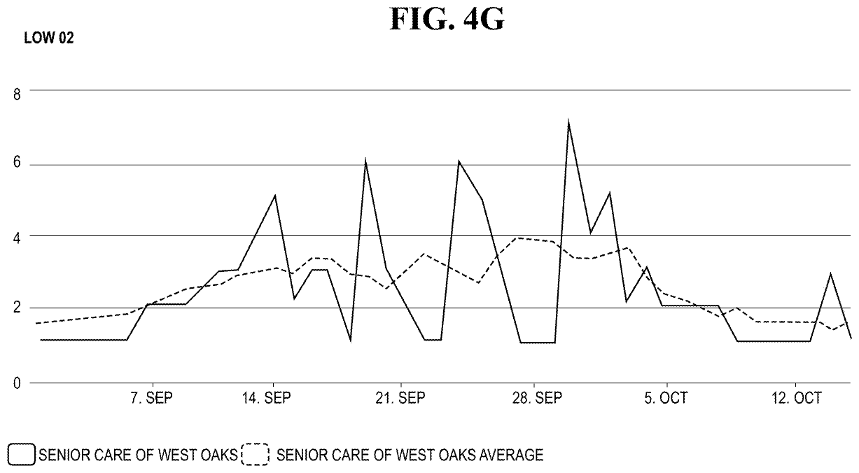

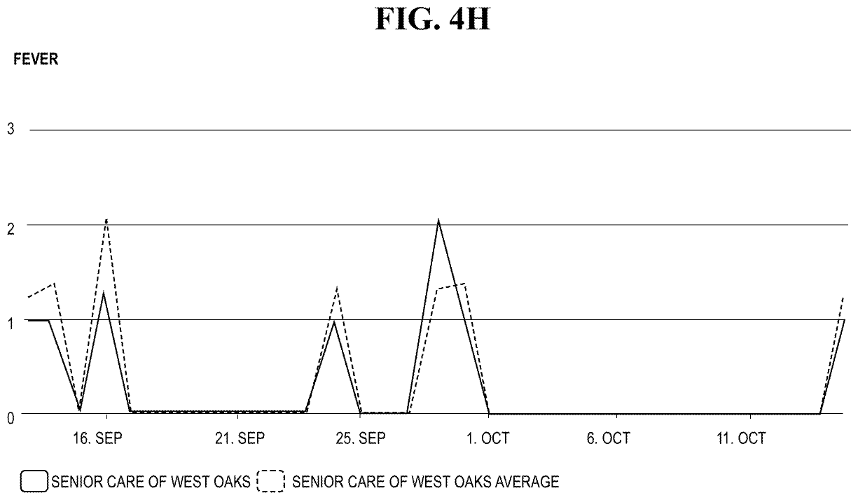

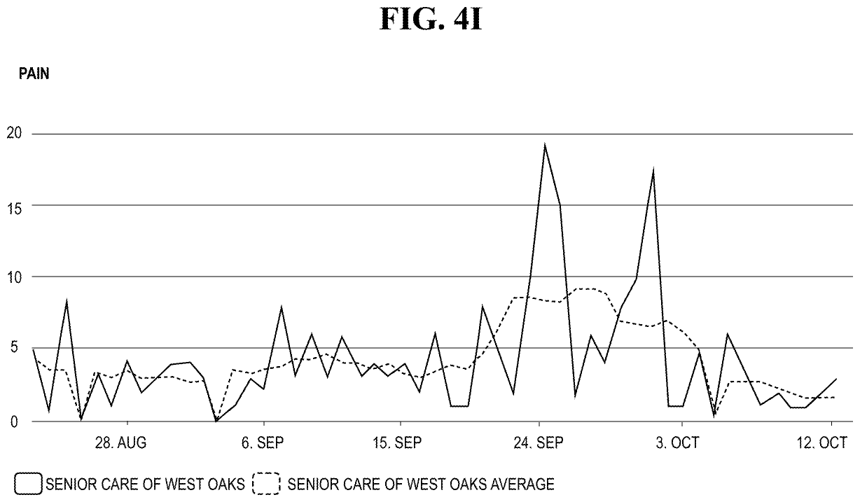

The present example demonstrates the use of system 10 and method 100 of the present invention in a SNF (“Facility #1) that contained a patient population of approximately 150 individuals. Patient data and metrics for each individual was collected/received and aggregated in system 10 as described in connection with method 100 above. The data was then processed and analyzed with reference to specific physiologically relevant criterion. As illustrated in FIG. 4, the physiologically relevant elements and/or criterion being monitored in this group of individuals were: hypertension, hypotension, tachycardia, bradycardia, hypoglycemia, hyperglycemia, O2 levels, pain (on a scale of 1 to 10), and temperature. The system 10 was able to aggregate the patient data and provide the data in an informative manner to the nurse practitioner, physician and other personnel at the facility. In particular, the system 10 was able to identify the occurrence of “triggering events” in individual patients relating to the monitored physiological criterion. This allowed the facility personnel to prioritize treatment and care for those patients.

As illustrated in FIG. 4, the system 10 allowed for identification of the occurrence of “triggering events” in the patient group as a whole to analyze the efficiency certain types of treatment and the medical attention provided at the facility to reduce the occurrence of “triggering events” among the group of patients. As shown in FIG. 4, through the use of system 10, the Facility #1 SNF was able to reduce the triggering incidences of both hypertension and hypotension over a two-month span in the patient population.

### EXAMPLE 5

### Skilled Nursing Home Facility #2

The present example demonstrates another use of system 10 and method 100 of the present invention in a different SNF that contained a patient population of approximately 50 individuals. Similar to the SNF Population #1, the system 10 was used to monitor the following physiologically relevant elements within the group of patients: hypertension, hypotension, tachycardia, bradycardia, hypoglycemia, hyperglycemia, O2 levels, pain (on a scale of 1 to 10), and temperature.

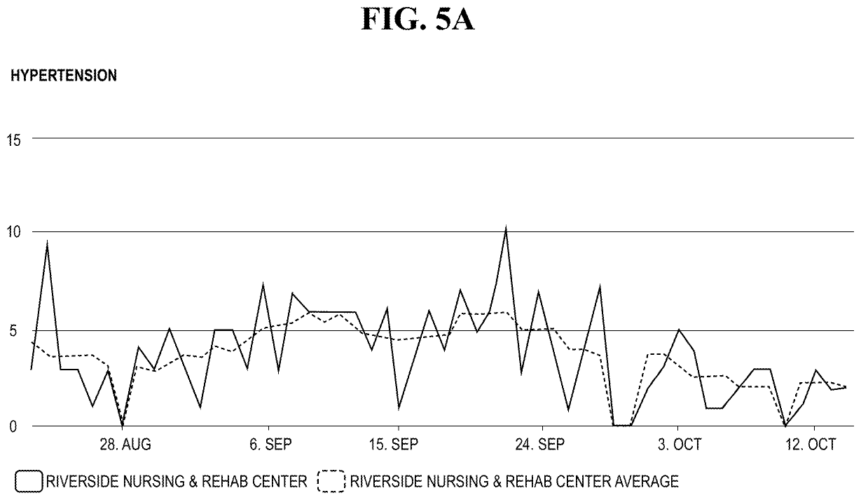

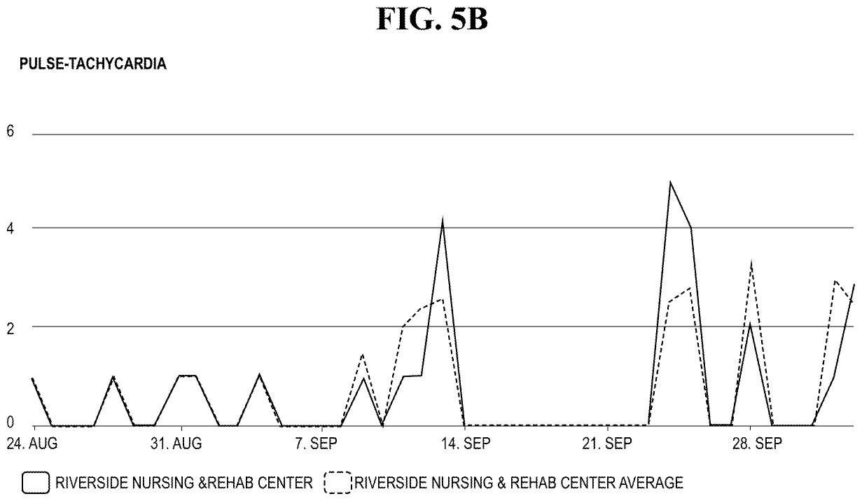

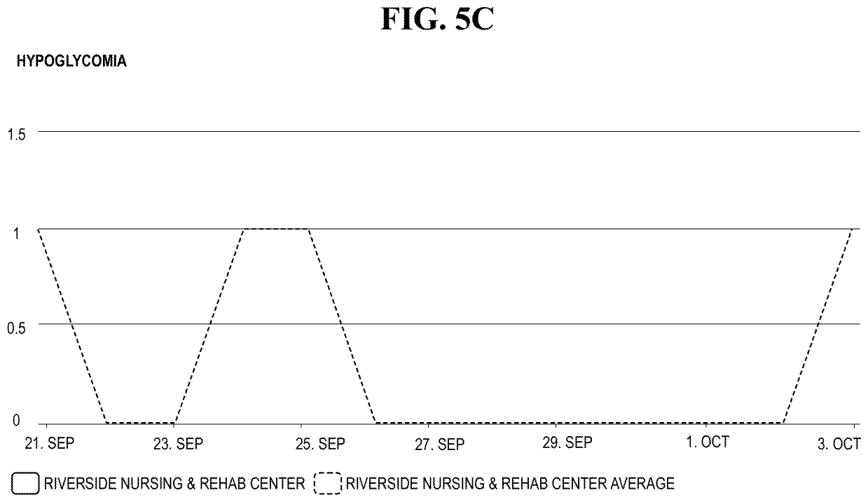

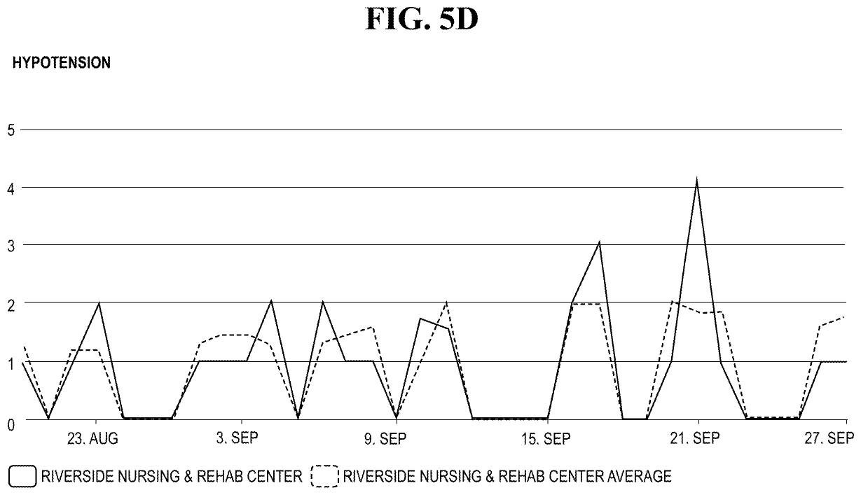

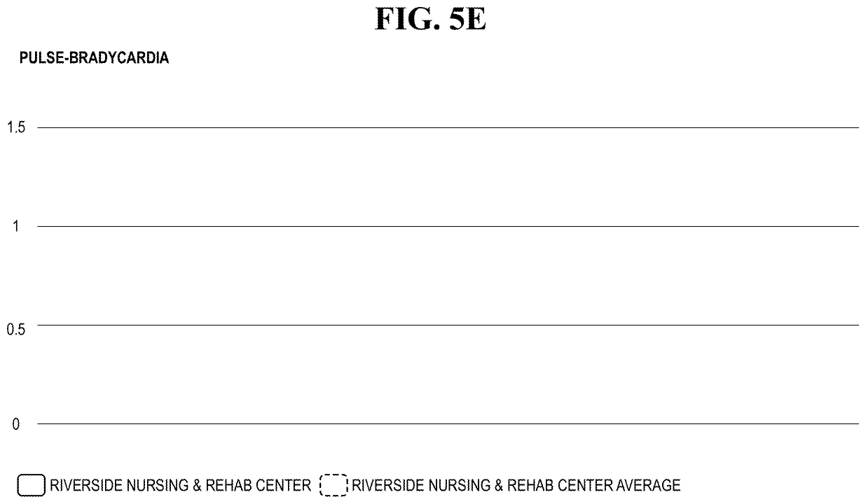

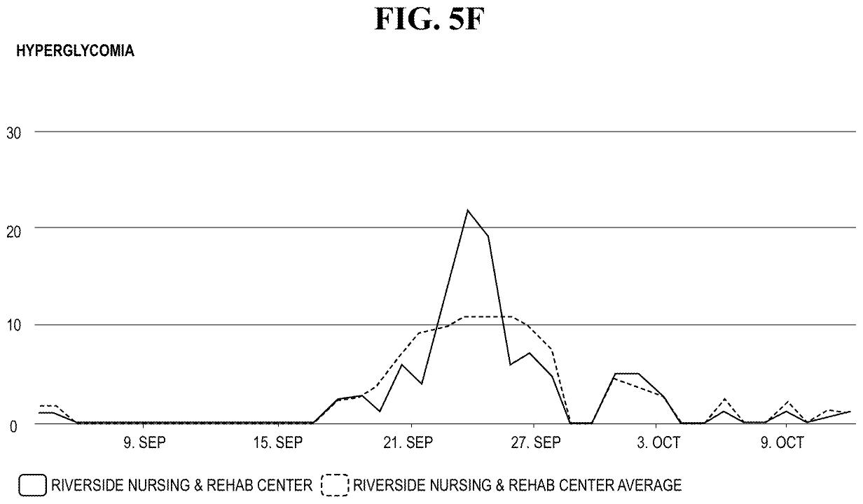

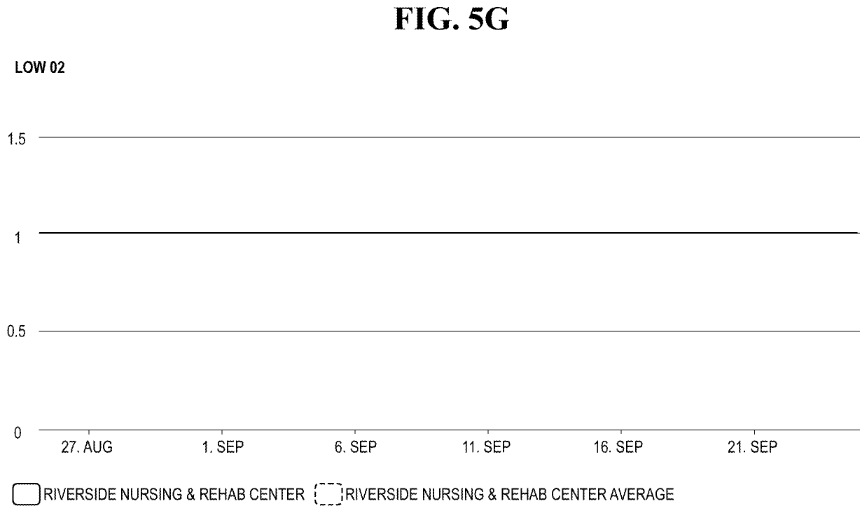

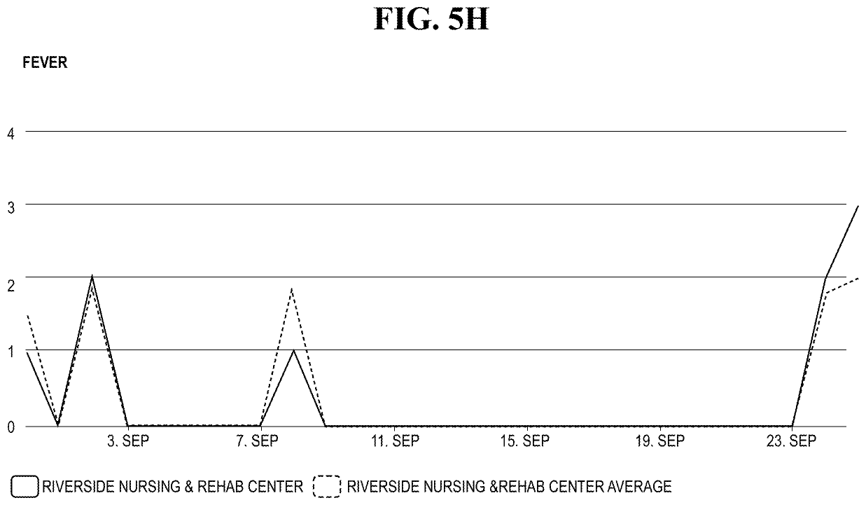

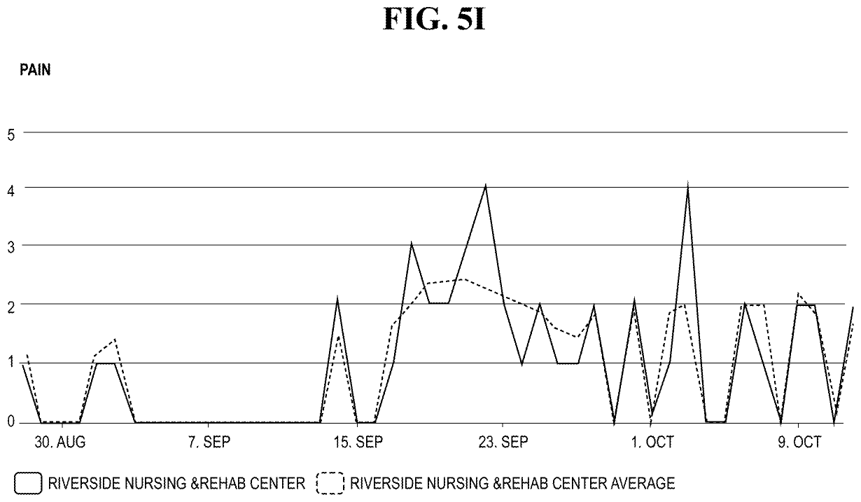

As illustrated in FIG. 5, system 10 allowed the medical personnel (physician, nurse practitioner, etc.) to efficiently monitor patient's health through the provided information and identification and triggering events. As further illustrated in FIG. 5, the use of system 10 was able to reduce the occurrence of hypertension as a triggering event in the patient population over a two-month span.

### EXAMPLE 5

### Skilled Nursing Home Facility #3

The present example demonstrates another use of system 10 and method 100 of the present invention in a different SNF that contained a patient population of approximately 40-50 individuals. Similar to the previous two SNF examples above, system 10 was utilized with the SNF facility #2 to monitor the following physiologically relevant elements within the group of patients: hypertension, hypotension, tachycardia, bradycardia, hypoglycemia, hyperglycemia, O2 levels, pain (on a scale of 1 to 10), and temperature. However, contrary to the previous two SNF examples, the medical personnel did not access and utilize the information generated and provided by system 10.

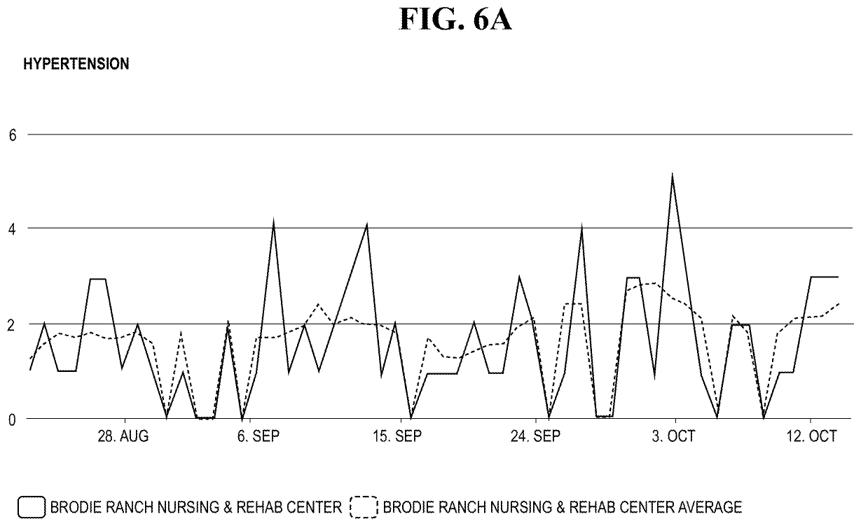

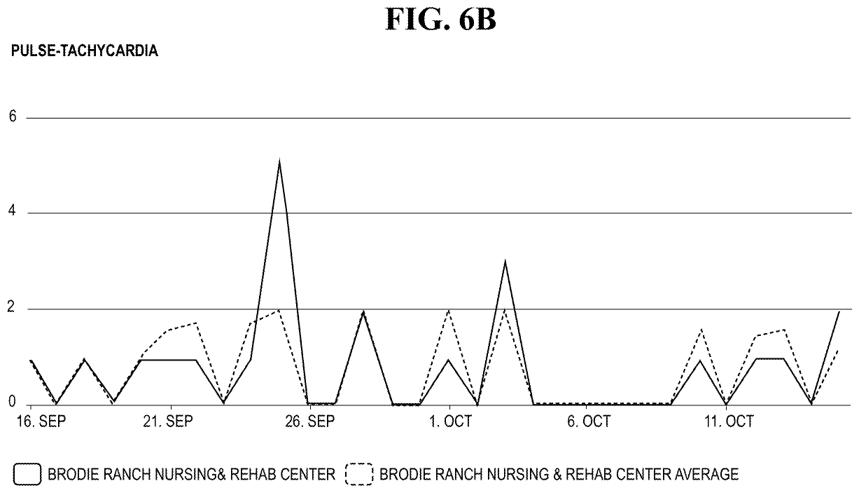

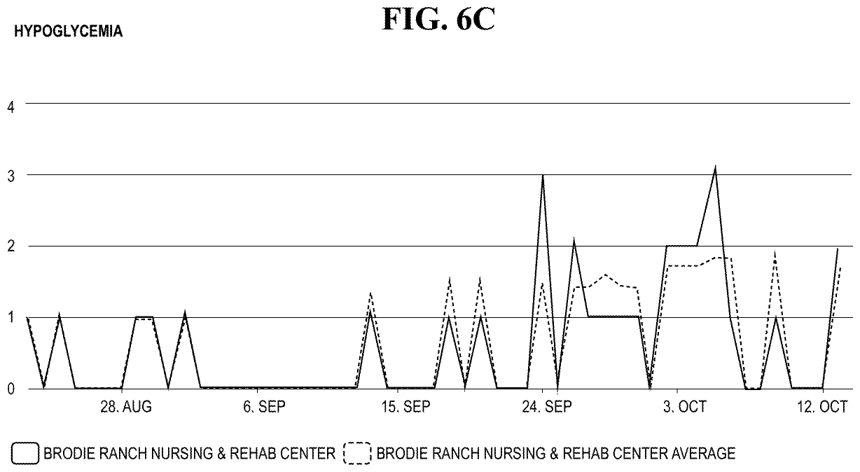

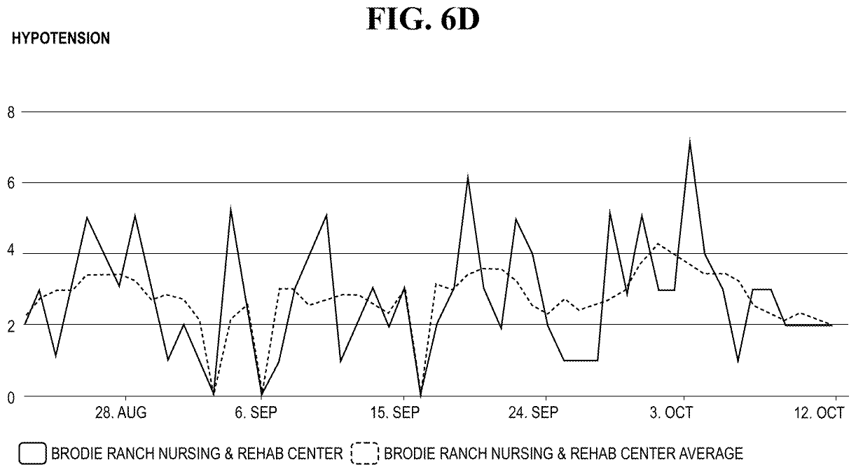

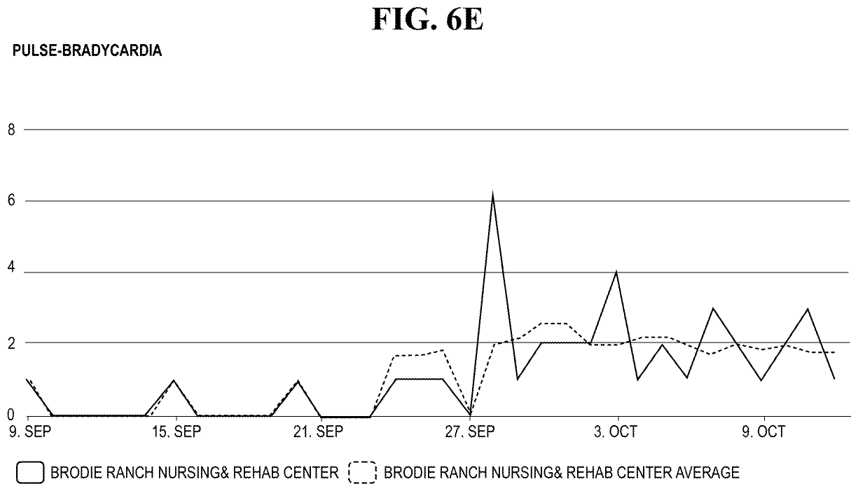

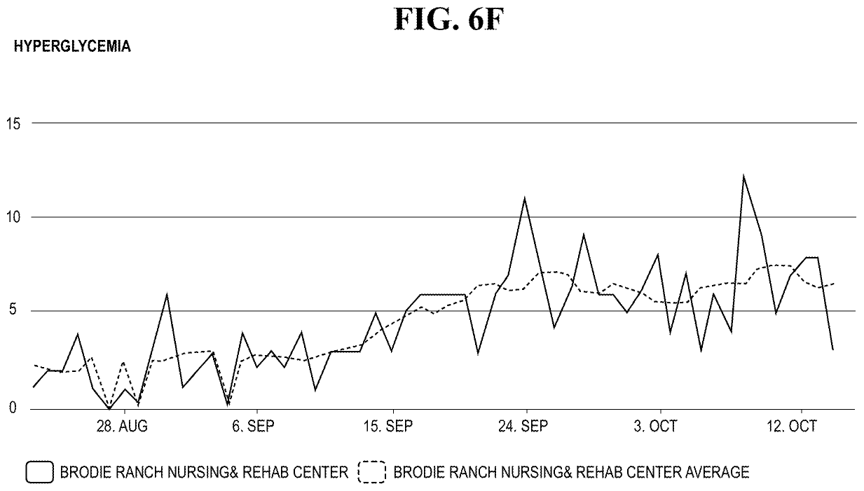

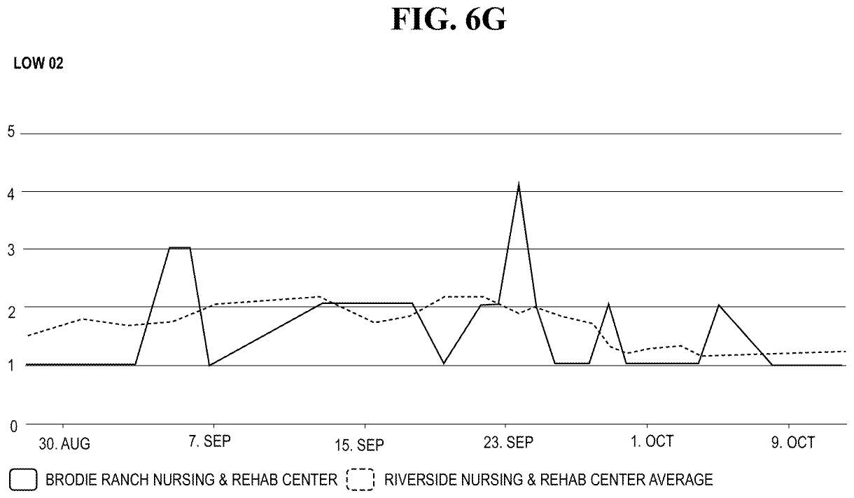

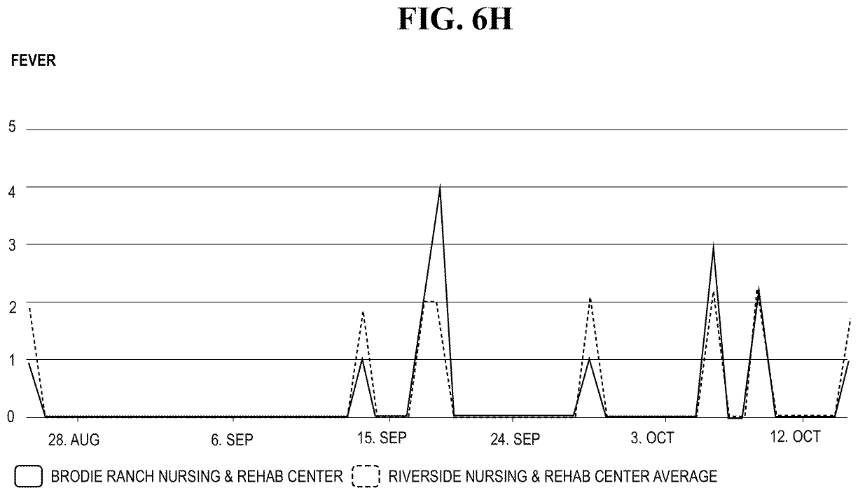

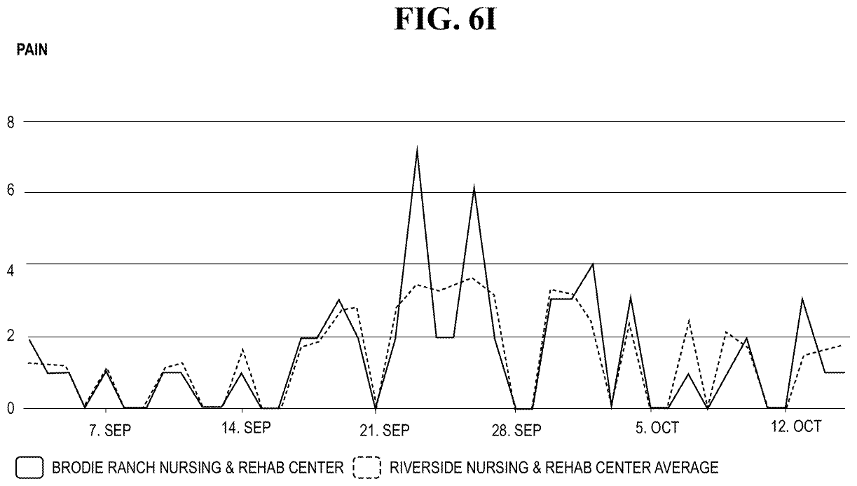

As illustrated in FIG. 6, the occurrence of triggering events did not decrease for any of the monitored physiological criterion and there was a lack of patient improvement as a result of the non-use of the system 10.

From the foregoing, it will be seen that this invention is one well adapted to attain all the ends and objects hereinabove set forth together with other advantages which are obvious and which are inherent to the structure. It will be understood that certain features and sub combinations are of utility and may be employed without reference to other features and sub combinations. This is contemplated by and is within the scope of the claims. Since many possible embodiments of the invention may be made without departing from the scope thereof, it is also to be understood that all matters herein set forth or shown in the accompanying drawings are to be interpreted as illustrative and not limiting.

The constructions described above and illustrated in the drawings are presented by way of example only and are not intended to limit the concepts and principles of the present invention. Thus, there has been shown and described several embodiments of a novel invention. As is evident from the foregoing description, certain aspects of the present invention are not limited by the particular details of the examples illustrated herein, and it is therefore contemplated that other modifications and applications, or equivalents thereof, will occur to those skilled in the art. The terms “having” and “including” and similar terms as used in the foregoing specification are used in the sense of “optional” or “may include” and not as “required”. Many changes, modifications, variations and other uses and applications of the present construction will, however, become apparent to those skilled in the art after considering the specification and the accompanying drawings. All such changes, modifications, variations and other uses and applications which do not depart from the spirit and scope of the invention are deemed to be covered by the invention which is limited only by the claims which follow.

We claim:

1. A method of stratifying a selected population of individuals to select for individuals having an identifiable triggering event, the method comprising the steps of:
a) aggregating patient data for each individual in the selected population of individuals, wherein the patient data includes physiological criterion;
b) analyzing the patient data to identify one or more triggering events associated with at least one selected physiological criterion in the patient data;
c) determining the individuals from the selected population of individuals having identified triggering events; and
d) generating a stratified list of individuals with identifiable triggering events and the associated physiological criterion.

2. The method of claim 1, wherein the step of analyzing patient data to identify triggering events includes determining whether patient data for the selected physiological criterion that is outside a baseline for the selected physiological criterion.

3. The method of claim 1 wherein the selected population of individuals are individuals in a skilled nursing facility.

4. The method of claim 1 wherein the selected physiological criterion is hypertension, hypotension, tachycardia, bradycardia, hypoglycemia, hyperglycemia, O₂ levels, pain (on a scale of 1 to 10), temperature, creatinine, or a combination thereof.

5. The method of claim 1 further comprising the step of displaying the stratified list of individuals to a user through a display device.

6. The method of claim 5 wherein the user is a health care provider, a physician, nurse practitioner, or physician's assistant.

7. An apparatus for performing the method of claim 1, the apparatus comprising:
a memory storage device storing a program of instructions;
a processor device receiving said program of instructions to configure said processor device to train a model comprising steps of: receiving data representing selected physiological criterion; and receiving data representing features of multiple persons within a defined group of individuals; and
a program of instructions to configure said processor device to display a selected group of individual physiological criterion values at a user interface.

8. The apparatus of claim 7, wherein the processor device is further configured to: perform an iterative algorithm on said selective physiological criterion and ranking the selective physiological criterion to identify an individual within said selected group having a triggering event as within a defined baseline for said selected population of individuals.

9. The apparatus of claim 8, wherein said iterative algorithm comprises a smoothing proximal gradient algorithm.

10. The apparatus of claim 9, wherein the processor device is further configured to: receive one or more of expert opinion data, and domain knowledge on risk association data.

11. The apparatus of claim 10, wherein the expert opinion data is data on a health care provider.

12. The apparatus of claim 7, wherein the selected physiological criterion is hypertension, hypotension, tachycardia, bradycardia, hypoglycemia, hyperglycemia, O₂ levels, pain (on a scale of 1 to 10), temperature, creatinine, or a combination thereof.

13. A method for generating a prioritized patient visit list for medical personnel, the method comprising the steps of:
receiving patient data for a selected group of patients;
aggregating, processing and analyzing the patient data based on a set of triggering criteria;
formatting the analyzed patient data for use by medical personnel;
generating a prioritized list of patients based on need of medical attention, wherein the prioritization is based on the incidence of triggering criteria for each patient from the selected group of patients; and
displaying the prioritized list of patients to medical personnel.

14. The method of claim 13, wherein the set of triggering criteria is determined by changes in one or more physiological criterion for each patient in the selected group of patients.

15. The method of claim 14, wherein the one or more physiological criterion includes hypertension, hypotension, tachycardia, bradycardia, hypoglycemia, hyperglycemia, O₂ levels, pain (on a scale of 1 to 10), temperature, creatinine, or a combination thereof.

16. The method of claim 13, wherein the patient data for each patient in the selected group of patients spans more than one time period.

17. The method of claim 13, wherein the step of aggregating, processing and analyzing the patient data based on a set of triggering criteria includes:
identifying specific physiological criterion; and
determining whether the physiological criterion exceeds a pre-determined threshold.

18. The method of claim 13, wherein the step of displaying the prioritized list of patients to medical personnel includes displaying the prioritized list on a mobile device.

19. The method of claim 13 further comprising displaying the patient data for at least one patient of the selected group of patients to the medical personnel.
